(19) United States
(12) Patent Application Publication
(10) Pub. No.: US 2022/0211614 A1
(43) Pub. Date: Jul. 7, 2022

(54) COMPOSITIONS AND IMPLANTABLE DEVICES

(71) Applicants: Commonwealth Scientific and Industrial Research Organisation, Acton, Australian Capital Territory (AU); The Florey Institute of Neuroscience and Mental Health, Parkville, Victoria (AU)

(72) Inventors: Nicholas WELCH, Noble Park ,Victoria (AU); Helmut THISSEN, Rowville , Victoria (AU); David WINKLER, Elwood ,Victoria (AU); Akhter HOSSAIN, Brunswick West ,Victoria (AU); John WADE, Canterbury ,Victoria (AU); Ross BATHGATE, Coburg ,Victoria (AU)

(21) Appl. No.: 17/595,750

(22) PCT Filed: Aug. 13, 2019
(86) PCT No.: PCT/AU2019/050847

§ 371 Date: Nov. 23, 2021

(30) Foreign Application Priority Data
May 24, 2019 (AU) 2019901773

(51) Int. Cl. A61K 9/00; A61B 5/263; A61K 38/17; A61K 35/39; A61K 35/28; A61K 47/34; A61K 45/06

(52) U.S. Cl. CPC A61K 9/0024; A61B 5/263; A61K 38/1796; A61K 45/06; A61K 35/28; A61K 47/34; A61K 35/39

(57) ABSTRACT

The present disclosure relates generally to a composition for inhibiting the foreign body response to an implantable device and an implantable device that comprises the composition. The disclosure also relates to methods for preparing a device for implantation in a subject.

### TECHNICAL FIELD

The present disclosure relates generally to a composition for inhibiting the foreign body response to an implantable device and an implantable device that comprises the composition. The disclosure also relates to methods for preparing a device for implantation in a subject.

### RELATED APPLICATIONS

This application claims priority to Australian Provisional Patent Application No. 2019901773 entitled “Compositions and implantable devices” filed 24 May 2019, the contents of which are incorporated herein by reference in their entirety.

### BACKGROUND

The current and next-generation of implantable biomedical devices are restricted by the inherent foreign body response (FBR) and subsequent fibrotic encapsulation of the implant. The FBR involves a complex series of cellular cascades initialized upon implantation of a (bio)material commencing with protein adsorption, followed by complement activation, cell adhesion, inflammation, macrophage fusion, fibrosis and encapsulation (Klopfleisch et al., J. Biomed. Mater. Res. 2017, 105:927-940). The resulting collagenous capsule that develops around the implant serves to physically and chemically isolate the foreign material. While this is an important mechanism to protect the body from adverse events associated with the accidental implantation of foreign objects, this response at the same time severely restricts the function of implantable medical devices by, for example, limiting the release and/or diffusion of molecules and gases, the detection of analytes, the provision of stimulatory signals, or the ability of the device to functionally interact with cells or tissue. Thus, for example, an encapsulated islet cell transplant (for addressing diabetes) can have reduced access to essential nutrients and gases which can lead to failure of the implant. Moreover, the release of insulin from the implant can be restricted. In another example, an indwelling continuous blood glucose monitor encapsulated as a result of the FBR can result in poor analyte diffusion and inaccurate sensing.

Thus, there is a need to develop compositions and materials that can be used in implantable devices and that inhibit fibrotic encapsulation and mitigate the FBR in order to extend the resident, functional lifetime of implantable devices.

### SUMMARY OF THE INVENTION

The present disclosure is predicated, at least in part, on the determination that sustained-release compositions comprising a RXFP1 agonist and a biocompatible polymer are surprisingly effective at inhibiting the foreign body response (FBR) to an implantable device, and in particular at inhibiting the development of a fibrous capsule around all or a portion of the implantable device. Inclusion of the composition in or on an implantable device can therefore significantly prolong the function of the implantable device, particularly in instances where the device actively interfaces or interacts with the body.

Thus, in one aspect, provided is an implantable device, comprising a composition comprising a relaxin family peptide receptor 1 (RXFP1) agonist and a biocompatible polymer adapted to facilitate sustained release of the RXFP1 agonist from the composition in vivo, wherein the implantable device is configured to detect a biological parameter, provide a stimulatory signal, facilitate tissue or organ repair and/or deliver a therapeutic agent, wherein the therapeutic agent is not an RXFP1 agonist.

The biological parameter may be, for example, an analyte, pH, temperature, light or an electrical signal. The stimulatory signal may be, for example, an electrical signal or an optical signal. In particular embodiments, the implantable device comprises an electrode. In further embodiments, where the implantable device is configured to deliver a therapeutic agent, the therapeutic agent may be a small molecule, polypeptide, peptide or polynucleotide.

In one embodiment, the implantable device is selected from among a biosensor, a cochlear implant, a neural implant, a peripheral nerve stimulator, a cellular implant, a nerve guide, a surgical mesh, a hernia repair implant, a retinal implant, a spinal cord stimulator, an optogenetic device, an optoelectric device, an infusion device and a drug depot.

In a further aspect therefore, provided is an implantable device, comprising a composition comprising a relaxin family peptide receptor 1 (RXFP1) agonist and a biocompatible polymer adapted to facilitate sustained release of the RXFP1 agonist from the composition in vivo, wherein the implantable device is selected from among a biosensor, a cochlear implant, a neural implant, a peripheral nerve stimulator, a cellular implant, a nerve guide, a surgical mesh, a hernia repair implant, a retinal implants, a spinal cord stimulator, an optogenetic device, an optoelectric device, an infusion device and a drug depot, and wherein the infusion device or the drug depot further comprises a therapeutic agent that is not an RXFP1 agonist.

In particular embodiments, where the implantable devices described above and herein are cellular implants, the cellular implant may comprise islet cells, mesenchymal cells or genetically-engineered cells.

In one embodiment, the composition is present as a coating on at least a portion of the surface of the implantable device.

The RXFP1 agonist may be, for example, relaxin (e.g. human relaxin 1, human relaxin 2 and/or human relaxin 3), a peptide analogue thereof (e.g. a single B chain peptide analogue, such as one comprising a truncation at the N-terminus of a B chain peptide analogue of relaxin and/or an extension at the C-terminus of a B chain peptide analogue of relaxin) or a small molecule. In particular embodiments, the relaxin or the peptide analogue thereof comprises an amino acid sequence set forth in any one of SEQ ID NOs:2-10. For example, the peptide analogue may be B7-33 having the amino acid sequence set forth in SEQ ID NO:10. In other embodiments, the RXFP1 agonist is a small molecule, such as ML290.

In some embodiments of the implantable devices of the present disclosure, the biocompatible polymer comprises a biodegradable polymer. In other embodiments, the biocompatible polymer comprises a non-biodegradable polymer. In particular embodiments, the biocompatible polymer is or comprises poly(lactic-co-glycolic acid) (PLGA), poly(ethylene glycol) (PEG), poly(ethylene oxide) (PEO), polylactide-polyglycolide homo- or co-polymers, poly(orthoester), polyglycolic acid (PGA), polylactic acid (PLA), polyurethane (PU), polyester, polycaprolactone (PCL), poly(hydroxy ethyl methacrylate), (PHEMA), polymethyl methacrylate (PMMA), polyethylene terephthalate (PET), poly(vinyl alcohol) (PVA), polysulfone, polytetrafluoroethylene (PTFE), poly(ethylene-co-vinyl acetate), polyethylene (PE), poly(propylene) (PP), poly(vinyl)chloride (PVC), polyetheretherketone (PEEK), polyvinylpyrollidone, polyacrylates, polymethacrylates, silicone, collagen, hyaluronic acid, polyanhydride, alginate, gelatin, albumin, collagen, polyamino acids, poly(amino alcohols), polyacrylamides, polymethacrylamides, zwitterionic polymers, or co-polymers or combinations of any of the foregoing. In one embodiment, the biocompatible polymer is selected from the group consisting of PLA, PGA, PLGA, and PCL.

In some examples of the implantable devices of the present disclosure, the composition comprises microspheres comprising the RXFP1 agonist and the biocompatible polymer. In further examples, the composition further comprises an additional biologically active agent (e.g. an anti-inflammatory agent, an anti-fibrotic agent, an antibiotic, a cytokine, and a pro-angiogenesis agent).

The functionality of the implantable devices in vivo may be prolonged (e.g. by at least about 20%, 40%, 60%, 80%, 100%, 150%, 200%, or more) compared to the functionality of a corresponding device that does not comprise the composition. In particular examples, release of the RXFP1 agonist from the composition when the device is implanted into a subject inhibits the foreign body response (FBR) to the device, e.g. inhibits by at least about 20% (e.g. by at least about 30%, 30%, 50%, 60%, 70%, 80%, 90% or more) when compared to the FBR when a corresponding device that does not comprise the composition is implanted into a subject. In further examples, the release of the RXFP1 agonist from the composition when the implantable device is implanted into a subject inhibits fibrotic encapsulation of all or a portion of the implantable device, e.g. fibrotic encapsulation is inhibited by at least about 20% when compared to the fibrotic encapsulation of a corresponding implantable device that does not comprise the composition.

A further aspect of the disclosure relates to a method for preparing a device for implantation in a subject, the method comprising providing an implantable device and coating at least a portion of the surface of the device with a composition, wherein:

the composition comprises a relaxin family peptide receptor 1 (RXFP1) agonist and a biocompatible polymer adapted to facilitate sustained release of the RXFP1 agonist from the composition in vivo; and

the implantable device is configured to detect a biological parameter, provide a stimulatory signal, facilitate tissue and/or organ repair, and/or deliver a therapeutic agent, wherein the therapeutic agent is not an RXFP1 agonist.

In some examples of the method, the biological parameter is selected from among an analyte, pH, temperature, light and an electrical signal. In further examples, the stimulatory signal is an electrical signal or an optical signal. In particular embodiments, the implantable device comprises an electrode. Where the implantable device is a configured to deliver a therapeutic agent, the therapeutic agent may be a small molecule, polypeptide, peptide or polynucleotide.

In particular embodiments of the method, the implantable device is selected from among a biosensor, a cochlear implant, a neural implant, a peripheral nerve stimulator, a cellular implant, a nerve guide, a surgical mesh, a hernia repair implant, a retinal implant, a spinal cord stimulator, an optogenetic device, an optoelectric device, an infusion device and a drug depot.

Thus, also provided is a method for preparing a device for implantation in a subject, the method comprising providing an implantable device and coating at least a portion of the surface of the device with a composition, wherein:

the composition comprises a relaxin family peptide receptor 1 (RXFP1) agonist and a biocompatible polymer adapted to facilitate sustained release of the RXFP1 agonist from the composition in vivo; and

the implantable device is selected from among a biosensor, a cochlear implant, a neural implant, a peripheral nerve stimulator, a cellular implant, a nerve guide, a surgical mesh, a hernia repair implant, a retinal implant, a spinal cord stimulator, an optogenetic device, an optoelectric device, an infusion device and a drug depot, and wherein the infusion device or the drug depot further comprises a therapeutic agent that is not an RXFP1 agonist.

In the methods of the present disclosure, the coating may comprise, for example, spray coating, dip coating, and/or mold coating.

Further aspects and illustrative embodiments of the invention are also described in the detailed description below.

### BRIEF DESCRIPTION OF THE FIGURES

Illustrative embodiments of the invention will now be described with reference to the following non-limiting figures in which:

FIG. 1 shows XPS of PLGA and PLGA+B7-33 coatings. High resolution C1s spectra of A) PLGA and B) PLGA+B7 33. Carbon contributions are shown with C1 (C—C), C2 (C—H), C3 (C—O), C4 (C═O) and C5 (O—C═O). Spectra are normalized to the C1 intensity at 285.0 eV. Quantification of C) the carbon contributions and D) the elemental composition of the coatings relative to the total carbon contribution. Trace contributions from Na, C1 and S were observed.

FIG. 2 shows an in vitro reporter gene assay for cyclic adenosine monophosphate (cAMP) activity in human embryonic kidney cells stably expressing the relaxin family peptide receptor 1 (HEK-RXFP1) demonstrates dose-dependent response of the applied peptide for PLGA+B7-33 surface coatings (squares), PLGA-free B7-33 controls (triangles) and B7-33 solution controls (circles). OD is optical density as measured for the colorimetric assay. Samples were prepared in triplicate and the data shown is the mean t standard deviation.

FIG. 3 shows cumulative peptide release from PLGA+B7 33 surface coatings over 61 days as measured using a BCA protein quantification kit. The optical density for each value was normalized to the B7-33 peptide control (i.e. maximum release). Samples were prepared in duplicate and the data shown is the mean t standard deviation.

FIG. 4 shows subcutaneous explants of PP coated with PLGA or PLGA+B7 33 at the 6-week time point. Fibrotic capsule formation is shown by (#).

FIG. 5 shows fibrotic capsule thickness from subcutaneous explants of PP coated with PLGA or PLGA+B7-33 at A) 2 week and B) 6 week time points. Data normalized to PLGA control. Twenty-three measurements were taken for duplicates of each sample type with the mean t standard error of the mean shown. **** P<0.0001.

FIG. 6 shows quantification of total cell count in the implant capsule (A-B) and cell area normalized to total capsule area (C-D) from subcutaneous explants PP coated with PLGA or PLGA+B7-33 at 2 week and 6 week time points. Single large area analysis conducted for duplicate samples with the mean t standard error of the mean shown. *P<0.05. NS, not significant.

FIG. 7 shows an in vitro reporter gene assay for cyclic adenosine monophosphate (cAMP) activity in human embryonic kidney cells stably expressing the relaxin family peptide receptor 1 (HEK-RXFP1), and demonstrates dose-dependent response of the applied agonist for coating compositions comprised of A) PLGA, B) polyurethane P80A, C) PCL, or D) polymer-free controls for RXFP1 agonists: relaxin (circles), B7-33 (squares) or ML290 (triangles). OD is optical density as measured for the colorimetric assay. Samples were prepared in triplicate and the data shown is the mean t standard deviation.

### DETAILED DESCRIPTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described.

The articles “a” and “an” are used herein to refer to one or to more than one (i.e., to at least one) of the grammatical object of the article. By way of example, “an RXFP1 agonist” means one RXFP1 agonist or more than one RXFP1 agonist.

As used herein, the term “about” refers to a quantity, level, value, dimension, size, or amount that varies by as much as 10% (e.g, by 10%, 9%, 8%, 7%, 6%, 5%, 4%, 3%, 2% or 1%) to a reference quantity, level, value, dimension, size, or amount.

As used herein, the term “biocompatible polymer” refers to a polymer material that, when introduced into a biological system (e.g., in vitro, ex vivo or in vivo), will have no, or substantially no, adverse impact on the biological system or on a part thereof. By “substantially no adverse impact” is to be understood to mean that the polymer may have some (negative and/or positive) impact on the biological system to which it comes into contact, but the extent of any such impact will be minimal and will not result, for example, in a reduction in the efficacy of the composition.

As used herein, the term “biologically active agent” refers to any molecule of synthetic or natural origin that is capable of eliciting a physiological response in a biological system, whether in vitro, ex vivo or in vivo.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

“Coating” as used herein refers to any temporary, semi-permanent or permanent layer, or layers, covering all or a portion of a surface of a substrate. For purposes herein, the coating involves the addition of a composition of the disclosure to the surface of a substrate, such as an implantable device. It includes any increase in thickness to the substrate. A coating may be applied as a liquid and solidified into a solid coating.

As used herein, “functionality” of an implantable device refers to the ability of the device to perform its primary purpose. Exemplary functionalities include, but are not limited to, detection of a biological parameter (e.g. an analyte, pH, temperature, light and/or an electrical signal), provision of a stimulatory signal (e.g. to a nerve or neuron), facilitation of tissue and/or organ repair (e.g. nerve regeneration), and delivery of a therapeutic agent. Reference to “prolonged” functionality means that the functionality (including a particular level or degree of functionality, e.g. 50%, 60%, 70%, 80%, 90% or more of the maximal or starting functionality of the device) is maintained over a longer duration, e.g. as measured in hours, days, weeks or months.

The term “implantable device” refers to any type of device that is totally or partly introduced, surgically or medically, into a subject's body or by medical intervention into a natural orifice of the subject's body, and which is intended to remain there after the procedure. The duration of implantation may be essentially permanent, i.e., intended to remain in place for the remaining lifespan of the subject; until the device biodegrades; or until the device is physically removed.

Reference to “relaxin” includes reference to natural human forms (including human relaxin 1, human relaxin 2, and human relaxin 3), animal forms, and synthetic forms. The term “relaxin” therefore includes relaxin polypeptides as isolated from vertebrates or relaxin polypeptides produced by recombinant techniques or techniques for peptide synthesis, and encompasses preprorelaxin, prorelaxin and relaxin, chimeric peptides with relaxin activity and relaxin variants or peptide analogues that differ from a wild-type relaxin by the addition, substitution, or deletion of one or more amino acid residues. In particular examples, peptide analogues of relaxin are single chain peptide analogues, e.g. single B chain analogues, such as B7-33. A relaxin for the purposes of the present disclosure exhibits anti-fibrotic activity. Typically, a relaxin will have anti-fibrotic activity that is at least or about 20%, 30%, 40%, 50%, 60%, 70%, 80%, 90%, 100% or more of that of human relaxin 2.

The term “relaxin family peptide receptor 1 agonist” or “RXFP1 agonist” refers to a molecule that binds and activates relaxin family peptide receptor 1 (RXFP1). Exemplary RXFP1 agonists include small molecules, polypeptides (e.g. RXFP1 agonist antibodies and antigen-binding fragments thereof) and peptides (e.g. relaxin and peptide analogues thereof). Typically, the RXFP1 agonist binds and activates human RXFP1, such as the human RXFP1 set forth in SEQ ID NO:1. Binding and activation of RXFP1 can be assessed using any methods known in the art, including in vitro assays such as an RXFP1 binding assay, cAMP activity assay or ERK1/2 phosphorylation assay, or using in vivo models to assess the ability of the RXFP1 agonist to inhibit fibrosis, i.e. to assess the anti-fibrotic activity of the agonist. Anti-fibrotic activity can be assessed using in vitro assays such assays to assess the induction of MMP expression, and in vivo models of fibrosis, as described herein, including models that assess the FBR to an implant. Typically, the anti-fibrotic activity of an RXFP1 agonist is such that the RXFP1 agonist can inhibit fibrosis by at least or about 20%, 30%, 40%, 50%, 60%, 70%, 75% 80%, 85%, 90%, 95% or more compared to fibrosis in the absence of the RXFP1 agonist. In other embodiments, an RXFP1 agonist has an anti-fibrotic activity that is at least or about 20%, 30%, 40%, 50%, 60%, 70%, 80%, 90%, 100% or more of that of human relaxin 2.

The term “subject” as used herein refers to an animal, in particular a mammal. Subjects include small animal subjects (e.g. mice, rats, rabbits, guinea pigs etc.), non-human primate subjects (e.g. chimpanzees) and human subjects. Typically, small animal or non-human primate subjects are used for pre-clinical assessment of a composition or implantable device of the present disclosure, while human subjects in need thereof are implanted with an implantable device of the present disclosure for therapeutic purposes.

The term “sustained release” as used herein refers to the release of an RXFP1 agonist from a biocompatible polymer (or a composition comprising the biocompatible polymer and RXFP1 agonist) subsequent to administration or delivery of the biocompatible polymer (or composition), such as in vivo in the context of an implantable device comprising a composition comprising the biocompatible polymer and RXFP1 agonist, whereby the rate of release of the RXFP1 agonist from the polymer is slower than would otherwise occur if the RXFP1 agonist was administered or delivered in the absence of the polymer. Sustained release will typically occur over a time period that is substantially longer than for rapid delivery. The sustained release of the RXFP1 agonist, as described herein, will typically provide a dose of the RXFP1 agonist over a longer period of time and therefore aid in prolonging the biological (e.g., therapeutic) effect provided by the RXFP1 agonist. In some embodiments, sustained release of the RXFP1 agonist occurs over a period of at at least or about 1, 5, 10, 15, 20, 25, 30, 35, 40, 45, 50, 55, 60, 65, 70, 75, 80, 85, 90, 95, 100, 120, 140, 160, 180, 200, 250, or 300 days or more.

The term “therapeutic agent” refers to any molecule of synthetic or natural origin that is capable of providing a therapeutic effect when delivered to a subject. The therapeutic effect may manifest, for example, as a reduction in the number of symptoms of a disease, condition or disorder, a reduction in the severity of one or more symptoms of a disease, condition or disorder, a reduction in a biological parameter (e.g. a reduction in the levels of an analyte), and/or an increase in a biological parameter (e.g. an increase in the levels of an analyte).

### Compositions

The present disclosure is predicted in part on the determination that a relaxin family peptide receptor 1 (RXFP1) agonist and a biocompatible polymer adapted to facilitate sustained release of the RXFP1 agonist from the composition in vivo is surprisingly effective at inhibiting the FBR to an implantable device comprising the composition, and in particular at inhibiting or preventing fibrotic encapsulation of all or a part of the device. The development of a fibrous capsule around all or a portion of the device, which is a hallmark of the FBR, may be completely inhibited or partially inhibited, the latter resulting in a fibrous capsule of reduced thickness compared to the fibrous capsule that would have developed in the absence of sustained-release of an RXFP1 agonist. Inclusion of the composition in or on an implantable device can therefore significantly prolong the function of the implantable device, particularly in instances where the device actively interfaces or interacts with the body, such as by detecting a biological parameter, providing a stimulatory signal, facilitating tissue or organ repair (e.g. by providing support or guidance for tissue or cell regeneration or growth) and/or delivering a therapeutic agent.

Thus, provided herein are compositions comprising a RXFP1 agonist and a biocompatible polymer adapted to facilitate sustained release of the RXFP1 agonist for use in implantable devices to inhibit the FBR to that device when the device is implanted in a subject, and in particular to inhibit or prevent fibrotic encapsulation of all or a part of the device. Inhibition of the FBR can be at least or about 20%, 25%, 30%, 25%, 40%, 45%, 50%, 55%, 60%, 65%, 70%, 75%, 80%, 85%, 90%, 95% or more compared to the FBR observed when a corresponding device that does not comprise the composition is implanted. Inhibition of the FBR may be measured by assessing inhibition of the fibrotic encapsulation of all or a part of the device. Fibrotic encapsulation (i.e. the formation of a fibrous capsule around all or a portion of the implantable device) may be inhibited by at least or about 20%, 25%, 30%, 25%, 40%, 45%, 50%, 55%, 60%, 65%, 70%, 75%, 80%, 85%, 90%, 95% or more compared to the fibrotic encapsulation of a corresponding device that does not comprise the composition. In some examples, the development of the fibrous capsule is completely inhibited over a particular duration (e.g. over days, weeks, months). In other examples, the development of the fibrous capsule is partially inhibited, such that the thickness of the fibrous capsule that does develop around all or a portion of the implantable device is less than, or is reduced compared to, the thickness of the fibrous capsule that develops around all or a portion of a corresponding implantable device that does not comprise the composition. The thickness of the fibrotic capsule can be reduced by at least or about 20%, 25%, 30%, 25%, 40%, 45%, 50%, 55%, 60%, 65%, 70%, 75%, 80%, 85%, 90%, 95%. The thickness of the fibrotic capsule can be assessed, for example, in pre-clinical studies, such as by implanting the device into a small mammal, such as a mouse, rat, rabbit, guinea pig, etc., or into a non-human primate, for a period of time then measuring the thickness of the fibrous capsule around all or a portion of the device. As would be appreciated, an inhibition of fibrous encapsulation can lead to prolonged functionality of the implantable device that comprises the composition, e.g. by at least or about 20%, 25%, 30%, 25%, 40%, 45%, 50%, 55%, 60%, 65%, 70%, 75%, 80%, 85%, 90%, 95%, 100%, 150%, 200%, 250% or more compared to the functionality of a corresponding device that does not comprise the composition.

Without being bound by theory or by a particular mode of action, the sustained release of the RXFP1 agonist may be attributed, at least in part, to the degradation of the biocompatible polymer over time and/or to the diffusion of the RXFP1 agonist into the environment from the biocompatible polymer in a manner that is independent of the degradation of the biocompatible polymer. By providing sustained release of the RXPF1 agonist, the effectiveness of the agonist to inhibit FBR over time can be enhanced. This is particularly true for agonists that have relatively short in vivo half-lives (e.g. recombinant human relaxin 2, which has a half-life of only 40 minutes, and ML290 which has a half-life of approximately 8 hours) and which would have a limited period of therapeutic effect (e.g. less than 1 or 2 days) if not formulated with a biocompatible polymer for sustained release. Conversely, when the RXFP1 agonist is provided in a composition described herein, the agonist is essentially protected from degradative processes until released from the polymer, and the continual release of the agonist over a sustained period provides for continuous therapeutic effect over that period.

In particular embodiments, the RXFP1 agonist is released from the biocompatible polymer (and thus the composition comprising the biocompatible polymer and the RXFP1 agonist) over a period of at least or about 1, 5, 10, 15, 20, 25, 30, 35, 40, 45, 50, 55, 60, 65, 70, 75, 80, 85, 90, 95, 100, 120, 140, 160, 180, 200, 250, or 300 days or more. In one embodiment, the composition provides sustained release of the RXFP1 agonist over a period of at least 7 days, at least 14 days, at least 28 days, or least 52 days following in vivo delivery, such as in the context of an implantable device comprising the composition. In further embodiments, at least or about 50% of the RXFP1 agonist is released from the polymer (or composition) over the first 12 hours, 24 hours, 36 hours, 48 hours, 60 hours, 72 hours, 84 hours or 96 hours. As would be appreciated by those skilled in the art, release rates may be adjusted by using differing polymers or combinations of polymers, different concentrations of polymer and/or RXFP1 agonist, and/or encapsulating the agonist in microspheres, nanospheres, etc. Strategies for adjusting the rate of release of an agent such as an RXFP1 agonist from a biocompatible polymer are well known to those skilled in the art (see e.g. Kamaly et al. Chem Rev. 2016, 116(4): 2602-2663).

The compositions typically comprise from 10 nM to 100 mM RXFP1 agonist, such as at least or about 10 nM, 100 nM, 200 nM, 300 nM, 400 nM, 500 nM, 600 nM, 700 nM, 800 nM, 900 nM, 1 μM, 10 μM, 20 μM, 30 μM, 40 μM, 50 μM, 60 μM, 70 μM, 80 μM, 90 μM, 100 μM, 120 μM, 140 μM, 160 μM, 180 μM, 200 μM, 250 μM, 300 μM, 350 μM, 400 μM, 450 μM, 500 μM, 550 μM, 600 μM, 650 μM, 700 μM, 750 μM, 800 μM, 850 μM, 900 μM, 950 μM, 1 mM, 5 mM, 10 mM, 20 mM, 30 mM, 40 mM, 50 mM, 50 mM, 70 mM, 80 mM, 90 mM or 100 mM RXFP1 agonist. As would be appreciated however, the precise amount of the RXFP1 agonist in the composition may depend on the identity of the antagonist, including its activity levels (e.g. its anti-fibrotic activity levels) and pharmacokinetic properties. The optimal amount of agonist in the composition can be determined empirically by those skilled in the art.

In some embodiments, from about 50 nM to about 5 μM, from about 500 nM to about 5 μM, from about 500 nM to about 50 μM, from about 5 μM to about 500 μM, from about 5 μM to about 50 μM, or from about 50 μM to about 500 μM RXFP1 agonist is released from the biodegradable polymer (and thus from the composition) over a period of least 7 days, at least 14 days, at least 28 days, or least 52 days following implantation of an implantable device comprising the composition of the present disclosure. In further embodiments, from about 1 ng to about 10 ng, from about 1 ng to about 100 ng, from about 10 ng to about 100 ng, from about 10 ng to about 1 μg, from about 100 ng to about 1 μg, from about 100 ng to about 10 μg, from about 1 μg to about 10 μg, from about 1 μg to about 100 μg, from about 10 μg to about 100 μg, or from about 100 μg to about 1 mg RXFP1 agonist is released from the biodegradable polymer (and thus from the composition) over a period of least 7 days, at least 14 days, at least 28 days, or least 52 days following implantation of an implantable device comprising the composition of the present disclosure. In further examples, at least or about 0.01, 0.05, 0.1, 0.2, 0.3, 0.4, 0.5, 0.6, 0.7, 0.8, 0.9, 1, 1.5, 2.0, 2.5, 3.0, 3.5, 4.0, 4.5, 5.0, 5.5, 6.0, 6.5, 7.0, 7.5, 8.0, 8.5, 9.0, 9.5, 10, 11, 12, 13, 14, 15, 20, 30, 40, 50, 60, 70, 80, 90 or 100 ng/day RXFP1 agonist is released from the biodegradable polymer (and thus from the composition) following implantation of an implantable device comprising the composition of the present disclosure.

The compositions comprising the RXFP1 agonist and a biocompatible polymer can be prepared by any suitable method, and such methods are well known to those skilled in the art. In some instances, a solution comprising the polymer (e.g. prepared using a solvent such as dimethylacetamide (DMAc), dimethylformamide (DMF), dimethylsulfoxide (DMSO), tetrahydrofuran (THF), acetone, etc.) and a solution comprising the RXFP1 agonist are simply admixed. In particular embodiments, the RXFP1 agonist is compounded or entrapped in a polymer microparticle or nanoparticle (e.g. a microsphere or nanosphere). Methods for preparing polymer-drug microparticles and nanoparticles are known and include, for example, oil-in-water or water-in-oil-in-water emulsion techniques, nanoprecipitation, spray-drying, salting-out, microfluidics and membrane extrusion emulsification (see e.g. Swida et al. Acta Biomed. 2018, 73:38-51).

### RXFP1 Agonists

RXFP1 agonists bind the G protein coupled-receptor (GPCR), Relaxin Family Peptide Receptor 1 (RXFP1), leading to stimulation of a multiple cell signalling pathways that include cyclic adenosine monophosphate (cAMP) and/or extracellular-signal-regulated kinases (ERK)1/2. For the purposes of the present disclosure, RXFP1 agonists bind and activate human RXFP1, such as set forth in SEQ ID NO: 1. Importantly, RXFP1 agonists suitable for inclusion in the compositions of the present disclosure exhibit anti-fibrotic activity.

Exemplary human RXFP1 (SEQ ID NO: 1):
MTSGSVFFYILIFGKYFSHGGGQDVKCSLGYFPCGNITKCLPQLLHCNGVD
DCGNQADEDNCGDNNGWSLQFDKYFASYYKMTSQYPFEAETPECLVGSVPV
QCLCQGLELDCDETNLRAVPSVSSNVTAMSLQWNLIRKLPPDCFKNYHDLQ
KLYLQNNKITSISIYAFRGLNSLTKLYLSHNRITFLKPGVFEDLHRLEWLI
IEDNHLSRISPPTFYGLNSLILLVLMNNVLTRLPDKPLCQHMPRLHWLDLE
GNHIHNLRNLTFISCSNLTVLVMRKNKINHLNENTFAPLQKLDELDLGSNK
IENLPPLIFKDLKELSQLNLSYNPIQKIQANQFDYLVKLKSLSLEGIEISN
IQQRMFRPLMNLSHIYFKKFQYCGYAPHVRSCKPNTDGISSLENLLASIIQ
RVFVWVVSAVTCFGNIFVICMRPYIRSENKLYAMSIISLCCADCLMGIYLF
VIGGFDLKFRGEYNKHAQLWMESTHCQLVGSLAILSTEVSVLLLTFLTLEK
YICIVYPFRCVRPGKCRTITVLILIWITGFIVAFIPLSNKEFFKNYYGTNG
VCFPLHSEDTESIGAQIYSVAIFLGINLAAFIIIVFSYGSMFYSVHQSAIT
ATEIRNQVKKEMILAKRFFFIVFTDALCWIPIFVVKFLSLLQVEIPGTITS
WVVIFILPINSALNPILYTLTTRPFKEMIHRFWYNYRQRKSMDSKGQKTYA
PSFIWVEMWPLQEMPPELMKPDLFTYPCEMSLISQSTRLNSYS

RXFP1 agonists are well known in the art and the compositions of the present disclosure can comprise any such agonist. Exemplary RXFP1 agonists include polypeptide, peptide and small molecule agonists.

In particular embodiments, the RXFP1 agonist is relaxin or a peptide analogue thereof. Exemplary relaxins include human relaxin 1 (H1), human relaxin 2 (H2) and, or human relaxin 3 (H3) (for review, see e.g. Patil et al., Br J Pharmacology, 2017, 174:950-961). Wild-type human relaxins are expressed as a pre-prohormone that is subsequently processed to a mature form that contains an A chain and B chain linked by two inter-chain disulphide bonds. Exemplary mature human relaxin sequences include:

H1 relaxin A chain:
(SEQ ID NO: 2)
RPYVALFEKCCLIGCTKRSLAKYC
H1 relaxin B chain:
(SEQ ID NO: 3)
VAAKWKDDVIKLCGRELVRAQIAICGMSTWS
H2 relaxin A chain:
(SEQ ID NO: 4)
QLYSALANKCCHVGCTKRSLARFC
H2 relaxin B chain:
(SEQ ID NO: 5)
DSWMEEVIKLCGRELVRAQIAICGMSTWS
H3 relaxin A chain:
(SEQ ID NO: 6)
DVLAGLSSSCCKWGCSKSEISSLC
H3 relaxin B chain:
(SEQ ID NO: 7)
RAAPYGVRLCGREFIRAVIFTCGGSRW

A number of relaxin analogues having anti-fibrotic activity are also known and are suitable for the compositions of the present disclosure. Such peptide analogues include two-chain and single chain (e.g. those having only a B chain) peptide analogues, and include analogues having N-terminal truncations and/or C-terminal extensions. Illustrative examples of relaxin peptide analogues include those disclosed in Hossain et al. (J. Biol. Chem., 2011, 286(43): 37555-37565) having C- or N-terminal truncations in the H2 A and/or B chain. These analogues include H2-(B3-29), H2-(B5-29), H2-(B7-29), H2-(B9-29), H2-(B1-28), H2-(B1-27), H2-(B1-26), H2-(B1-25), H2-(B1-24), H2-(B1-23), H2-(B7-25), H2-(B8-25), H2-(B7-24), H2-(B8-24), H2-(A2-24)(B7-24), H2-(A3-24)(B7-24), H2-(A4-24)(B7-24), H2-(A5-24)(B7-24), H2-(A7-24)(B7-24), H2-(A9-24)(B7-24), H2-(A-Z-5-24)(B7-24), H2-(A-Z-5-24)(B7-24), H2-(A-Z-7-24)(B7-24), H2-(A5-24)(B7-24) (acid), and/or H2-(A4-24)(B7-24) (acid), with numbering relative to the amino acid positions of the A and B chains set forth in SEQ ID Nos: 4 and 5. Illustrative examples of relaxin peptide analogues also include those described in WO2015157829, and in particular the single chain analogues comprising the amino acid sequence VIKLCGRELVRAQIAICGMSTWS (SEQ ID NO:8); VIKLCGRELVRAQIAICGMSTWSKRSL (SEQ ID NO:9); or VIKLSGRELVRAQIAISGMSTWSKRSL (SEQ ID NO:10). As described in WO2015157829 and Hossain et al. (Chem. Sci., 2016, 7, 3805-3819), the peptide analogue B7-33 set forth in SEQ ID NO: 10 is particularly effective as a RXFP1 agonist and is contemplated for inclusion in the compositions of the present disclosure.

Relaxins for use in accordance with the present disclosure also include relaxin fusion polypeptides, such as described in WO2013004607, comprising a relaxin A chain, a linker and a relaxin B chain (i.e. A chain—linker-B chain).

In other embodiments, the RXFP1 agonist is a small molecule. Illustrative examples of small molecule RXFP1 agonists include those described in U.S. Pat. No. 9,452,973. In one example, the RXFP1 agonist is a compound or pharmaceutically acceptable salt thereof having the formula:

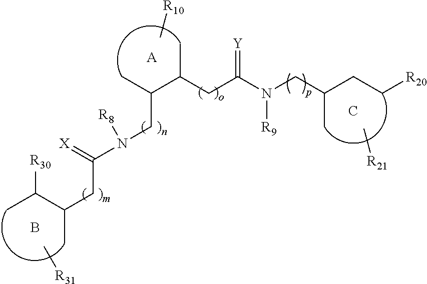

where

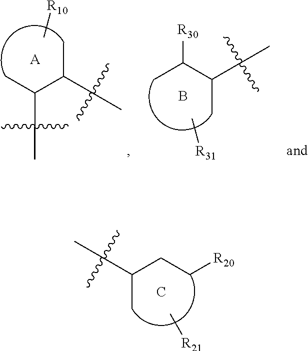

are each phenyl;
m, n, o, and p are integers independently chosen from 0.
1, and 2 and each of

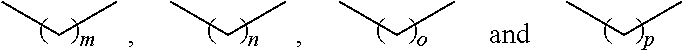

is unsubstituted or substituted with one or more substituents independently chosen from halogen, hydroxyl, C₁-C₂alkyl, and C₁-C₂alkoxy;
X and Y are independently chosen from O and S;
R₈ and R₉ are independently chosen from hydrogen and C₁-C₄alkyl;
R₁₀, R₂₁, and R₃₁ are each 0 to 3 substituents independently chosen from hydroxyl, halogen, nitro, cyano, amino, C₁-C₄alkyl, C₁-C₄alkocy, mono- and di-(C₁-C₂alkyl)amino-, C₁-C₂haloalkyl, and C₁-C₂haloalkoxy;
R₂₀ is C₁-C₁₀haloalkyl, C₁-C₁₀haloalkoxy, —SR₇, —SOR₇, or —SO₂R₇, where R is C₁-C₁₀carbyhdryl or C₁-C₁₀haloalkyl;
R₃₀ is hydrogen or R₃₀ is C₁-C₈carbhydryloxy or C₁-C₈carbhydrylthio-each or which is substituted with 0 to 3 substituents independently chosen from hydroxyl, halogen, nitro, cyano, C₁-C₄alkyl, C₁-C₄alkoxy, C₁-C₄haloalkyl, and C₁-C₄haloalkoxyl.

In another example, the small molecule RXFP1 agonist is ML290 (Xiao et al. Nature Comm, 2013, 4:1-7; Xiao et al. 2012, Probe Reports from the NIH Molecular Libraries Program) having the formula:

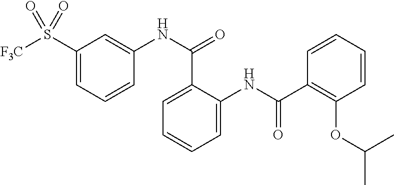 [SMILES: CC(C)Oc1ccccc1C(=O)Nc1ccccc1C(=O)Nc1cccc(S(=O)(=O)C(F)(F)F)c1]

Other small molecule RXFP1 agonists contemplated for use herein include those described in McBride et al. Sci Rep. 2017, 7: 10806.

It will be appreciated that the above list of RXFP1 agonists is non-limiting, and other agonists suitable for formulation with a biocompatible polymer in the compositions of the present disclosure are known. Moreover, those skilled in the art can identify or generate other RXFP1 agonists (e.g. small molecule agonists or peptide agonists). Methods for assessing the ability of a molecule (such as a small molecule or peptide) to bind RXFP1 and act as an agonist are well known, and include, for example, in vitro assays such as RXFP1 binding assays to assess the ability of the molecule to bind RXFP1, cAMP activity assays to assess the ability of the molecule to increase cAMP levels, ERK1/2 phosphorylation assays to assess the ability of the molecule to induce ERK1/2 phosphorylation, and matrix metalloproteinase (MMP) assays to assess the ability of the molecule to increase MMP expression (see, for example, Examples 1 and below, and Hossain et al. Chem. Sci., 2016, 7, 3805-3819). Methods of assessing the ability of the molecule to inhibit fibrosis in vivo, i.e. to assess the in vivo anti-fibrotic activity of the molecule, are also known and include, but are not limited to, animal models of myocardial infarction- or isoprenaline hydrochloride-induced heart failure, both of which are characterised by the development of fibrosis, animal models of OVA-induced acute allergic airway disease (AAD)), and/or assessment of the FBR to an implant (see, for example, Examples 1 and 5, below, and Hossain et al. Chem. Sci., 2016, 7, 3805-3819). In some examples, transgenic mice expressing the human RXFP1 gene instead of the mouse RXFP1 gene are used to assess ability of the molecule to act as a RXFP1 agonist in vivo and inhibit fibrosis (Kaftanovskaya et al. J Endocr Soc. 2017, 1(6): 712-725).

Typically, a molecule that is suitable as a RXFP1 agonist for the purposes of the present disclosure can inhibit fibrosis by at least or about 20%, 25%, 30%, 35%, 40%, or more preferably at least or about 45%, 50%, 55%, 60%, 65%, 70%, 75%, 80%, 85%, 90%, 95% or more. In further embodiments, RXFP1 agonist will exhibit an anti-fibrotic activity that is comparable to that of human relaxin 2, although may be less or more. For example, the RXFP1 agonist can exhibit anti-fibrotic activity that is at least or about 20%, 30%, 40%, 50%, 60%, 70%, 80%, 90%, 100%, 120%, 140%, 160%, 180%, 200%, 300%, 400%, 500% or more of that of human relaxin 2.

### Biocompatible Polymers

Suitable biocompatible polymers that are adapted to facilitate sustained release of the RXFP1 agonist from a composition comprising the polymer and agonist are known in art, and any such polymer can be employed herein.

The biocompatible polymer can be a synthetic or a natural (i.e., naturally-occurring) polymer. Illustrative examples of suitable natural polymers include proteins such as albumin, collagen, gelatin and prolamins, for example, zein, and polysaccharides such as alginate, cellulose derivatives and polyhydroxyalkanoates, for example, polyhydroxybutyrate.

The biocompatible polymer may be a biodegradable polymer, a non-biodegradable polymer, or substantially non-biodegradable polymer. It would be understood, however, that it is generally desirable that the biocompatible polymer is biodegradable, or substantially biodegradable, so as to avoid or minimise the impact the polymer may otherwise have on a biological system over time.

In one embodiment, the biocompatible polymer is a biodegradable polymer. Suitable biodegradable polymers will be known to persons skilled in the art, illustrative examples of which are polypeptides, alginates, chitosan, starch, collagen, silk fibroin, polyurethanes, polyacrylic acid, polyacrylates, polyacrylamides, polyesters, polyolefins, boronic acid functionalised polymers, polyvinylalcohol, polyvinyl pyrrolidone, poly(lactic acid), polyether sulfone, inorganic polymers, and a combination of any of foregoing. Thus, in an embodiment disclosed herein, the biodegradable polymer is selected from the group consisting of polypeptides, alginates, chitosan, starch, collagen, silk fibroin, polyurethanes, polyacrylic acid, polyacrylates, polyacrylamides, polyesters, polyolefins, boronic acid functionalised polymers, polyvinylalcohol, polyvinyl pyrrolidone, poly(lactic acid), polyether sulfone, inorganic polymers.

The biodegradable polymer can be selected to degrade over a time period ranging from 1 day to 1, 2, 3 or more years, e.g. 7 days to 52 weeks, 7 days to 26 weeks, 7 days to 20 weeks, or 7 days to 16 weeks. It will be understood that the choice of polymer may depend on the intended use, e.g. the type of implantable device comprising the composition and the expected time period in which the device remains implanted in a subject. In some embodiments, a synthetic polymer may be preferred. In other embodiments, a natural polymer may be preferred. Other illustrative examples of suitable polymers include poly(lactic acid), poly(glycolic acid), poly(lactic acid-co-glycolic acids), polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); poly(glycolide-co-caprolactones); polycarbonates such as tyrosine polycarbonates; polyamides (including synthetic and natural polyamides), polypeptides, and poly(amino acids); polyesteramides; other biocompatible polyesters; poly(dioxanones); poly(alkylene alkylates); hydrophilic polyethers; polyurethanes; polyetheresters; polyacetals; polycyanoacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), polyvinyl alcohols, polyvinylpyrrolidone; poly(alkylene oxides) such as polyethylene glycol (PEG); derivativized celluloses such as alkyl celluloses (e.g., methyl cellulose), hydroxyalkyl celluloses (e.g., hydroxypropyl cellulose), cellulose ethers, cellulose esters, nitrocelluloses, polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), as well as derivatives, copolymers, and blends thereof. Derivatives include polymers having substitutions, additions of chemical groups and other modifications to the polymeric backbones described above routinely made by those skilled in the art. Natural polymers, including proteins such as albumin, collagen, gelatin, prolamins, such as zein, and polysaccharides such as alginate and pectin, are also contemplated.

In particular examples, the polymer is selected from among poly(lactic-co-glycolic acid) (PLGA), poly(ethylene glycol) (PEG), poly(ethylene oxide) (PEO), polylactide-polyglycolide homo- or co-polymers, poly(orthoester), polyglycolic acid (PGA), polylactic acid (PLA), polyurethane (PU), polyester, polycaprolactone (PCL), poly hydroxy ethyl methacrylate, (PHEMA), polymethyl methacrylate (PMMA), polyethylene terephthalate (PET), poly(vinyl alcohol) (PVA), polysulfone, polytetrafluoroethylene (PTFE), Poly(ethylene-co-vinyl acetate), polyethylene (PE), poly(propylene) (PP), poly(vinyl)chloride (PVC), polyetheretherketone (PEEK), polyvinylpyrollidone, polyacrylate, (hydroxyethyl)methacrylate, silicone, collagen, hyaluronic acid, polyanhydride, alginate, gelatin, albumin, collagen, polyamino acids, poly(amino alcohols), or co-polymers or combinations of any of the foregoing. In one example, the biocompatible polymer is or comprises PLA, PGA, PLGA, or PCL.

In one preferred embodiment, the biocompatible polymer comprises poly(lactic acid). In a particular embodiment, the poly(lactic acid) is poly(lactic-co-glycolic acid) (PLGA). In some embodiments, the poly(lactic-co-glycolic acid) is poly(D,L-lactide-co-glycolide). An exemplary poly(lactic-co-glycolic acid) is one with a lactide:glycolide ratio of about 50:50, 65:35, 75:25, or 85:15. The poly(lactic-co-glycolic acid), for example poly(D,L-lactide-co-glycolide), may have an average molecular weight (Mw) of 7 kDa to about 250 kDa. In some examples, the poly(D,L-lactide-co-glycolide) has a Mw from about 76 kDa to about 115 kDa, from about 50 kDa to about 75 kDa, from about 66 kDa to about 107 kDa, from about 30 kDa to about 60 kDa, from about 7 kDa to about 17 kDa, or from about 6 kDa to about 10 kDa.

### Other Agents and Additives

The compositions may suitably comprise an additional biologically active agent. The biologically active agent may be, for example, a functional small molecule (e.g. a drug) or larger biomolecules (e.g., proteins, peptides, enzymes, polynucleotides including oligonucleotides, etc.). Non-limiting examples of additional biologically active agents that may be present in the compositions of the present disclosure include an anti-inflammatory agent (e.g. dexamethasone (DEX), alpha melanocyte-stimulating hormone (α-MSH), heparin, interleukin-1 receptor antagonist (IL-1Ra), superoxide dismutase mimetics, curcumin, vitamin E, etc.), a growth factor (e.g. nerve growth factor (NGF), vascular endothelial growth factor (VEGF), epidermal growth factor (EGF)), a hormone, a pro-angiogenesis agent and an antibiotic.

### Implantable Devices

The present disclosure provides implantable devices comprising a composition described above and herein, i.e. a composition comprising RXFP1 agonist and a biocompatible polymer adapted to facilitate sustained release of the RXFP1 agonist from the composition in vivo. As a result of this sustained release of the RXFP1 agonist, the FBR to the device when implanted in a subject is inhibited. In particular, the development of the fibrous capsule around all or a portion of the device, which is a hallmark of the FBR, is inhibited. Inhibition of the FBR can be at least or about 20%, 25%, 30%, 25%, 40%, 45%, 50%, 55%, 60%, 65%, 70%, 75%, 80%, 85%, 90%, 95% or more compared to the FBR observed when a corresponding device that does not comprise the composition is implanted. Inhibition of the FBR can be measured by assessing inhibition of fibrotic encapsulation of all or a part of the device, and such inhibition may be at least or about 20%, 25%, 30%, 25%, 40%, 45%, 50%, 55%, 60%, 65%, 70%, 75%, 80%, 85%, 90%, 95% or more compared to the fibrotic encapsulation of a corresponding device that does not comprise the composition. In some examples, the development of the fibrous capsule is completely inhibited over a particular duration (e.g. over days, weeks, months following implantation into a subject). In other examples, the development of the fibrous capsule is partially inhibited, such that the thickness of the fibrous capsule that does develop around all or a portion of the implantable device is less than, or is reduced compared to, the thickness of the fibrous capsule that develops around all or a portion of a corresponding implantable device that does not comprise the composition. The thickness of the fibrotic capsule can be reduced by at least or about 20%, 25%, 30%, 25%, 40%, 45%, 50%, 55%, 60%, 65%, 70%, 75%, 80%, 85%, 90%, 95%. The thickness of the fibrotic capsule can be assessed, for example, in pre-clinical studies, such as by implanting the device into a small mammal, such as a mouse, rat, rabbit, guinea pig, etc., or into a non-human primate, for a period of time then measuring the thickness of the fibrous capsule around all or a portion of the device.

Particularly contemplated herein are implantable devices that actively interface or interact with cells, tissue or fluid in a subject. The function of such devices is especially sensitive to the development of a fibrous capsule around all or a portion of the device, and in particular around the portion of the device that actively interfaces or interacts with cells, tissue or fluid. For example, the development of a fibrous capsule that impedes the detection of a biological parameter, impedes the provision of a stimulatory signal, impedes tissue and/or organ repair, and/or impedes the delivery of a therapeutic agent by an implantable device, has a significant impact on the function of that device. Consequently, implantable devices of the present disclosure that comprises a RXFP1 agonist and a biocompatible polymer adapted to facilitate sustained release of the RXFP1 agonist typically have prolonged or extended functionality compared to a corresponding device that does not comprise the composition. In some examples, functionality is extended by at least or about 20%, 30%, 40%, 50%, 60%, 70%, 80%, 90%, 100%, 110%, 120%, 150%, 200%, 250%, 300%, 400%, 500% or more.

In particular embodiments, therefore, the implantable devices of the present disclosure are configured to detect a biological parameter, provide a stimulatory signal, facilitate tissue and/or organ repair, and/or deliver a therapeutic agent, wherein the therapeutic agent is not an RXFP1 agonist.

Biological parameters that can be detected by the implantable device include, for example, an analyte, pH, temperature, light and/or an electrical signal. Illustrative examples of analytes that the implantable device may be configured to detect include glucose, fructose and other vicinal diols; α-hydroxy acids; β-keto acids; oxygen; carbon dioxide; various ions such as zinc, potassium, or hydrogen; toxins; minerals; and hormones. Thus, implantable device of the present disclosure include biosensors (including intravascular, transdermal or intracranial sensors). Biological parameters that can be detected may also include electrical signals such as those generated by neuronal or nerve cells.

Implantable devices that are configured to provide a stimulatory signal may provide an electrical signal, such as for stimulating a neuron or nerve. In other examples, the implantable device that is configured to provide a stimulatory signal provides an optical signal (i.e. a light signal, such as used in optogenetic or optoelectronic devices) for stimulating a neuron or other cell (see e.g. Mickle et al. Nature, 2019, 565:361-365). Thus, implantable devices of the present disclosure that are configured to provide a stimulatory signal include cochlear implants, retinal implants, neural implants, peripheral nerve stimulators, spinal cord stimulators, and optogenetic or optoelectronic devices.

As would be appreciated from the above, in some embodiments implantable devices of the present disclosure, including cochlear implants, retinal implants, neural implants, peripheral nerve stimulators, spinal cord stimulators, optogenetic or optoelectronic devices, and biosensors, comprise an electrode.

In other embodiments the implantable devices of the present disclosure are configured to facilitate tissue and/or organ repair. Such devices include, but are not limited to, nerve guides and surgical scaffolds, urogynecologic surgical mesh implants and hernia repair implants.

In still further embodiments, the implantable devices are configured to deliver a therapeutic agent other than an RXFP1 agonist. These implantable devices can be used treat a disease or condition that is treatable by administration of the therapeutic agent, but is not treatable by the administration of the RXFP1 agonist, i.e. the primary function of the device is not the treatment of a fibrotic condition that is treatable by administration of the RXFP1 agonist; the primary function of the implantable device is the treatment of a disease or condition that is treatable by administration of the therapeutic agent other than an RXFP1 agonist. For example, the implantable device may be an implantable drug depot or a continuous infusion device that delivers a therapeutic agent, e.g. an anti-cancer agent, a pain-relief agent, an anti-microbial agent, an anti-inflammatory agent, an anti-arrhythmic agent, glucose, insulin, etc., so as to treat cancer, pain, a microbial infection, an inflammatory condition, a heart condition, diabetes, etc. In other examples, the implantable device is a cellular implant that secretes the therapeutic agent, such as an islet cell implant that secretes insulin or a genetically-engineered cellular implant that secretes a recombinant therapeutic protein. Cellular implants are typically encapsulated in a polymeric semi-permeable membrane (e.g. a polymeric membrane comprising poly(ether-sulfone), poly(lactic-co-glycolic acid), the copolymer of polyacrylonitrile and polyvinyl chloride, polyurethane, polysulfone, polypropylene, polyvinylidene difluoride, polytetrafluoroethylene, alginate, collagen, gelatin, agarose, cellulose sulphate, etc.) so as to allow diffusion of the therapeutic agent that is produced by the implant out of the implant, and also so as to allow nutrients and oxygen to diffuse into the implant (for review, see e.g. Lathuilere et al. Int J Mol Sci. 2015, 16(5): 10578-10600). In some embodiments, the composition of the present disclosure forms this semi-permeable membrane encapsulating the cellular implant. In other embodiments, the composition of the present disclosure is present as a coating on all or a part of the encapsulated cellular implant.

An implantable device of the present disclosure therefore includes, but is not limited to, a biosensor, a cochlear implant, a neural implant, peripheral nerve stimulator, a cellular implant, a nerve guide, a surgical mesh, a retinal prosthesis, a spinal cord stimulator, an infusion device, a drug depot, an optogenetic device and an optoelectronic device.

The composition comprising the RXFP1 agonist and the biocompatible polymer may be integrated into a substrate of the device, or may be present as a coating on all or a portion of the surface of the device. In some embodiments, it is desirable that not all of the device or the surface of the device comprises the composition, so as to allow or promote a FBR at a site adjacent to where the composition is not present. Allowing or promoting the formation of a fibrous capsule on a select portion of the device can help anchor the device to the tissue. As would be appreciated however, the portion of the device that does not comprise the composition is one that is not essential to the function of the device, e.g. does not comprise an electrode or sensor, and/or is not directly involved in providing a stimulatory signal or detecting a biological parameter, does not provide a scaffold for tissue or organ repair, and/or does not facilitate delivery of a therapeutic agent.

Methods for assessing the functionality of a device and the duration of functionality may differ between devices, depending on the nature of the device. For example, assessing the functionality of a biosensor may involve determining how long the device accurately detects or senses an analyte; assessing the functionality of a cellular implant may involve determining how low the implant secretes a therapeutically effective amount therapeutic agent, such as insulin or a recombinant protein, etc. Determining an appropriate means for assessing the functionality of a device is well within the skill of a practitioner.

### Manufacturing Processes

The present disclosure also provides a method for preparing a device for implantation in a subject, the method comprising providing an implantable device and coating at least a portion of the surface of the device with a composition of the present disclosure (i.e. a composition comprising a RXFP1 agonist and a biocompatible polymer adapted to facilitate sustained release of the RXFP1 agonist from the composition in vivo); wherein the implantable device is configured to detect a biological parameter, provide a stimulatory signal, facilitate tissue or organ repair and/or deliver a therapeutic agent, wherein the therapeutic agent is not an RXFP1 agonist; and/or wherein the implantable device is selected from among a biosensor, a cochlear implant, a neural implant, a peripheral nerve stimulator, a cellular implant, a nerve guide, a surgical mesh, a hernia repair implant, a retinal implant, a spinal cord stimulator, an optogenetic device, an optoelectric device, an infusion device and a drug depot, and wherein the infusion device or the drug depot further comprises a therapeutic agent that is not an RXFP1 agonist.

Also provided is a method for enhancing the in vivo function of an implantable device, comprising providing an implantable device and coating at least a portion of the surface of the device with a composition of the present disclosure (i.e. a composition comprising a RXFP1 agonist and a biocompatible polymer adapted to facilitate sustained release of the RXFP1 agonist from the composition in vivo); wherein the implantable device is configured to detect a biological parameter, provide a stimulatory signal, facilitate tissue or organ repair and/or deliver a therapeutic agent, wherein the therapeutic agent is not an RXFP1 agonist; and/or wherein the implantable device is selected from among a biosensor, a cochlear implant, a neural implant, a peripheral nerve stimulator, a cellular implant, a nerve guide, a surgical mesh, a hernia repair implant, a retinal implant, a spinal cord stimulator, an optogenetic device, an optoelectric device, an infusion device and a drug depot, and wherein the infusion device or the drug depot further comprises a therapeutic agent that is not an RXFP1 agonist.

Methods for coating a substrate with a polymeric composition are well known in the art and include, but are not limited to, spray coating, dip coating, and/or mold coating techniques.

Unless defined otherwise, all technical and scientific terms used herein have the same meanings as commonly understood by one of ordinary skill in the art to which this invention belongs. Although any materials and methods similar or equivalent to those described herein can be used to practice or test the present invention, the preferred materials and methods are now described.

All references, including any patent or patent application cited in this specification are hereby incorporated by reference to enable full understanding of the invention. Nevertheless, such references are not to be read as constituting an admission that any of these documents forms part of the common general knowledge in the art, in Australia or in any other country.

The invention will now be described with reference to the following Examples which illustrate some preferred aspects of the present invention. However, it is to be understood that the particularity of the following description of the invention is not to supersede the generality of the preceding description of the invention and that various other modifications and/or alterations may be made without departing from the spirit of the present invention, as disclosed herein.

### EXAMPLES

### Example 1

### Materials and Methods

### Materials

Greiner Bio-One polypropylene microplates (655201) were purchased from Griener Bio-One GmbH (Germany). Microscope slide tray plates (1345-40) were purchased from Mobitech GmbH (Germany). Resomer® RG 756 S poly(D,L-lactide-co-glycolide) (PLGA; ester terminated, lactide:glycolide 75:25, Mw 76,000-115,000) and the QuantiPro™ BCA Assay Kit (QPBCA-1KT) were purchased from Sigma (Australia). Dimethylformamide (DMF) was purchased from Merck (Australia).

### B7-33 Peptide Synthesis

B7-33 was synthesized as reported previously (Hossain et al. Chem. Sci. 2016, 7:3805-3819). Briefly, the peptide was manually assembled on solid phase as C-terminal amide, cleaved by trifluroacetic acid after synthesis, analysed and purified using RP-HPLC via an analytical and preparative column respectively. The molecular mass of B7-33 was determined by electrospray ionisation mass spectroscopy (ESI MS)—B7-33: m/z 2986.4 [M+H]⁺, calculated 2986.59. The peptide content was quantified by Direct Detect® spectrometer, an infrared-based protein quantitation system. The purity of B7-33 was determined to be 97%.

### PLGA+B7-33 Coatings

PLGA solutions (5 mg/mL) were prepared in DMF with the desired concentration of the B7-33 antifibrotic peptide (100, 10, 1, 0.1, 0.01, 0.001, 0.0001 μM). Peptide solution in DMF (in the absence of PLGA) were also prepared as controls. The desired solution was dispensed (50 μL) into polypropylene well plates and dried under vacuum to <0.01 mbar to remove residual solvent. Wells were then sealed with an adhesive film seal and stored at 4° C. until use. Solution concentrations were chosen such that addition of 100 μL of media to the dried surface would yield the required final peptide concentration. Similarly, B7-33 (100 μM to 10 μM), whole H2 relaxin (1 nM to 0.1 fM) and ML290 (100 μM to 10 μM) were prepared in PLGA, PCL or polyurethane P80A (5 mg/mL) to make polymer coatings. Dichloromethane was chosen as the solvent for PCL. XPS samples were prepared by cleaning silicon wafers (1×1 cm) in a sonication bath (30 min) with 2% RBS detergent (Sigma), and 2% ethanol in Milli-Q water (18.2 MΩ·cm). Wafers were then rinsed with excess ethanol and water before being dried with a stream of nitrogen. The PLGA (5 mg/mL) solution containing B7-33 (100 μM) and was then dispensed (50 μL) to each wafer and before drying under vacuum.

### Peptide Quantification

PLGA+B7-33 coatings were prepared in well plates as above incorporating 10 μM of B7-33. Peptide solution in DMF (in the absence of PLGA) were also prepared as controls. 200 μL of phosphate-buffered saline (PBS) was added to each well and incubated at 37° C. At the desired time points (1-61 days) 150 μL was sampled from the appropriate wells in triplicate and dispensed into a storage well plate sealed with an adhesive microplate film seal and stored at 4° C. until the completion of the experiment. The peptide control (in the absence of PLGA) was sampled on the final day. The QuantiPro™ BCA Assay Kit was then used as per the manufactures instructions for all sampled time points in triplicate. The plate was analysed in a plate reader at 562 nm and the mean and standard deviation of the optical density (OD) value for each triplicate calculated in Microsoft Excel. Each sample value was then normalized relative to the peptide control sample value.

### HPLC-MS and MALDI-TOF MS Measurements

PLGA B7-33 well plate coatings (prepared at 10 μM as above) were incubated with 100 μL of Milli-Q water at 37° C. for 6 h. 50 μL aliquots were removed from wells and characterized by HPLC-MS and MALDI-TOF MS. Firstly, mass spectrometric analyses were performed on a Thermo Scientific Q Exactive mass spectrometer fitted with a HESI-II ion source. Positive ion electrospray mass spectra were recorded in an appropriate mass range at 140,000 mass resolution. The probe was used with 0.3 ml/min flow of solvent. The nitrogen nebulizing/desolvation gas used for vaporization was heated to 100° C. in these experiments. The sheath gas flow rate was set to 25 and the auxiliary gas flow rate to 7 (both arbitrary units). The spray voltage was 3.8 kV and the capillary temperature was 300° C. UHPLC Conditions: Chromatographic analysis was performed using a Dionex 3000 UHPLC system. The Chromatographic conditions were as follows: Column: Thermo Hypersil Gold C18 (50×2.1 mm, 1.9 μm particle size); Mobile Phase A: 98% water and 2% methanol with 0.1% formic acid; Mobile Phase B: Methanol with 0.1% formic acid; Gradient: 100% Mobile Phase A to 100% Mobile Phase B in 8 min and then held for 5 min; Flow Rate: 300 μl/min; Column Temperature: 30° C.; Sample Injection Volume: 10 μl. MALDI measurements were performed on a Bruker ultrafleXtreme MALDI-TOF/TOF mass spectrometer. For MALDI analysis, the 6 h time point for the peptide control (i.e. no PLGA) was compared to freshly prepared peptide in Milli-Q water. Buffer and media solutions were avoided to reduce the complexity, and to maintain the integrity, of the peptide mass spectrum. Sample preparation: From the extracted water solutions, the B7-33 peptide samples were then dissolved in 85% acetonitrile, 15% water and 0.1% trifluoroacetic acid (TA85) solvent at a concentration of 1 mg/mL. A stock solution of the matrix was prepared by dissolving 10 mg of α-Cyano-4-hydroxycinnamic acid (HCCA) matrix name in 1 mL TA85. The sample and matrix solutions were combined in the ratio of 100 μL to 10 μL, respectively. A 1 μL aliquot of the mixture was then applied to the PAC (Pre-spotted Anchor Chip) MALDI plate and air-dried at ambient temperature (20° C.). Measurements were performed at an acceleration voltage of 20 kV, in positive ion reflectron mode. Suitable values for laser power, gain and laser shots were determined for each sample to produce the best quality data (best resolution, reduced fragmentation and ion statistics).

HEK-RXFP1 cAMP Reporter Gene Assay

B7-33 activity at RXFP1 was assessed using a cAMP reporter gene assay as previously described (Scott et al. J. Biol. Chem. 2006, 281:34942-3495) with slight modification. PLGA B7-33 well plate coatings, and peptide controls in the absence of PLGA (dried peptide), were compared to solubilized B7-33 solution standards (10 μM to 10 μM) and H2 relaxin (10 nM). Wells were seeded (25,000 cells per well, 100 μL) with dispersed HEK-RXFP1-pCRE reporter cells and incubated for 6 hours. Cells were then lysed and the reporter gene stimulated β-galactosidase activity was measured using a colorimetric substrate to evaluate the relative cAMP responses. Ligand-induced cAMP stimulation was expressed as a % of maximal response to H2 relaxin. Each data point was measured in triplicate and each experiment conducted independently at least three separate times.

### In Vivo PLGA+B7-33 Samples

Polypropylene (PP) samples (1×1 cm, 1 mm thick) were cut from microscope slide tray lids and sonicated in 2% RBS detergent, 2% ethanol, water solution for 1 h and then rinsed with excess ethanol and Milli-Q water before drying with a stream of nitrogen. To improve wettability, PP samples were subject to air plasma in a custom built reactor for 30 s each side (frequency 200 kHz, load power 20 W, and initial chamber pressure 0.6 mbar). Plasma-treated PP samples were then dip coated in a DMF solution of PLGA (5 mg/mL) with or without B7-33 peptide (100 μM) before being dried under vacuum to <0.01 mbar. Samples were then placed in well plates, vacuum sealed and gamma sterilized at 15 kGy (Steritech, Australia). Samples were then stored at 4° C. until implantation.

### X-Ray Photoelectron Spectroscopy

Surface analysis was conducted using an AXIS Ultra DLD x-ray photoelectron spectrometer (Kratos Analytical, Inc., Manchester, UK) fitted with a monochromated Al Ka source supplying an x-ray power of 180 W (15 kV×12 mA). The hemispherical analyser operated in the fixed transmission mode, and the aperture limited the analysis area to 0.3×0.7 mm. The total pressure inside the analysis chamber was maintained below 10⁻⁸ mbar during analysis. All survey spectra were acquired at a pass energy of 160 eV. High resolution C1s spectra were acquired at a pass energy of 40 eV. All samples were analysed at an emission angle of 0° relative to the surface normal. X-ray photoelectron spectroscopy (XPS) spectra quantification and data processing were performed using CASAXPS software version 2.3.16 (Casa Software, Ltd., Teignmouth, UK). All elements present were identified from the survey spectra, the atomic concentrations were calculated using integral peak intensities, and the relative sensitivity factors were supplied by the manufacturer. Binding energies are referenced to the aliphatic hydrocarbon peak at 285.0 eV.

### In Vivo Assessment

All animal husbandry, housing and experimental procedure were performed as approved by Monash Medical Centre Animal Ethics Committee A (2017/05). Female C57BL/6 mice aged 8-16 weeks were randomly divided into 2 experimental groups namely (i) PLGA and (ii) PLGA+B7-33. The mice were anaesthetized with 3% w/v Isoflurane® and carprofen (5 mg/kg body weight) was used as analgesia. A longitudinal skin incision was performed in the lower abdomen and scaffold of 1×1 cm size each was implanted into each animal using Ethicon 6-0 suture. All animals were housed in the animal house at Monash Medical Centre according to the National Health and Medical Research Council of Australia guidelines for the care and use of laboratory animals. Mice were housed individually for 1 week followed by 4 mice per cage until endpoint. Animals were euthanized in a CO₂ chamber and scaffold tissue areas were harvested at 2 or 6 weeks (2 mice/group/time-point) for histological analysis. The harvested tissues were fixed using formalin and embedded in paraffin blocks followed by cutting into 8 μm sections on poly-L-lysine slides. The sections were stained using Hematoxylin and Eosin stains by Monash Histology as per established protocols.

### Statistics

GraphPad Prism (ver 7.04) was used to calculate statistical significance using the accompanying t-test analysis function. Statistics were calculated on technical replicates with biological duplicates for each group and time point.

### Example 2

PLGA+B7-33 Surface Coating Preparation and Characterization

A simple method for the incorporation and release of the anti-fibrotic peptide B7-33 from a polymer coating was designed. PLGA is a well-known and characterized biodegradable polymer with tuneable release characteristics. It was reasoned that a PLGA coating would offer a mechanism to deliver the B7-33 peptide locally to the implant site at biologically-relevant concentrations. Subsequently, the peptide would mediate the deposition and degradation of collagen and therefore offer the ability to reduce fibrotic capsule formation in vivo. PLGA+B7-33 coatings were prepared on polypropylene (PP) model implants using a dip-coat approach.

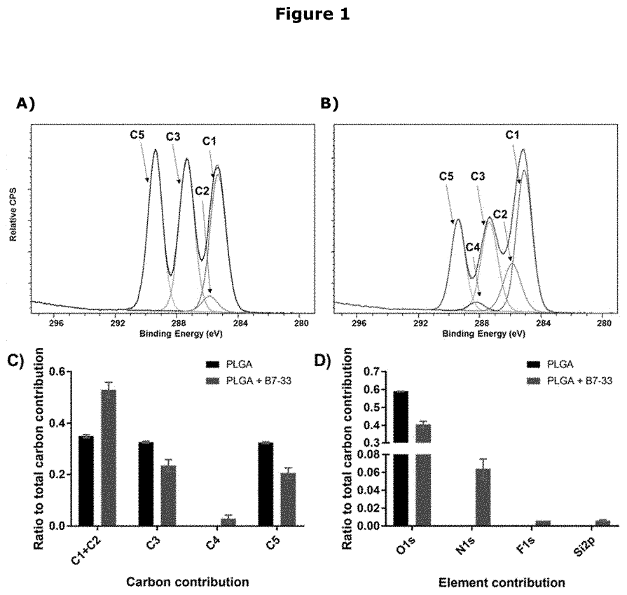

X-ray photoelectron spectroscopy (XPS) was used to characterize the surface composition of silicon wafers coated with PLGA in the absence and presence of B7-33. The high resolution C1s spectra (FIG. 1A) confirmed the presence of the PLGA coating with the expected, approximately equal contributions from C1+C2 (C—C and C—H, 285.0 and 286.5 eV), C3 (C—O, 287.3 eV) and C5 (O—C═O, 289.5 eV). For the PLGA+B7-33 coatings (FIG. 1B), the ratio of C1+C2 to both C3 and C5 were reduced relative to the PLGA coatings as would be expected with an increase in C—H contributions from the peptide. Quantification of the carbon contributions is shown in FIG. 1C. As expected, when B7-33 was incorporated in the PLGA coating, a reduction in the oxygen-to-carbon (0/C) ratio and an increase in the nitrogen-to-carbon ratio (N/C) for the PLGA+B7-33 relative to PLGA coatings alone was observed, confirming the presence of the peptide (FIG. 1D). A small amount of fluorine was noted, residual from the preparation of the peptide with trifluoroacetic acid (TFA) salt.

### Example 3

### PLGA+B7-33 Surface Coating Preparation and Characterization

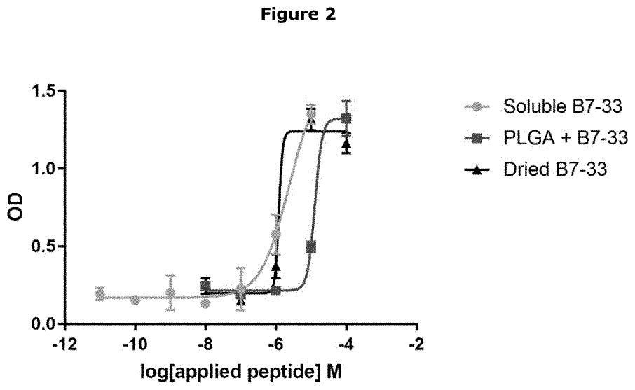

Satisfied that the method described above could be used to dip-coat substrates with PLGA+B7-33 coatings, it was next investigated whether the B7-33 peptide retained its activity against the native relaxin RXFP1 receptor after coating processing. For ease of handling, PLGA+B7-33 coatings were prepared into polypropylene 96 well plates. The coatings were interrogated with an in vitro reporter gene assay for cyclic adenosine monophosphate (cAMP) activity in human embryonic kidney cells stably expressing the relaxin family peptide receptor 1 (HEK-RXFP1) (FIG. 2). The B7-33 peptide solution control (grey) and the PLGA-free dried peptide control (black) exhibited equivalent EC50 at a concentration of 1 μM, consistent with earlier work (Hossain et al. Chem. Sci., 2016 7:3805-3819). Importantly, this indicated that the process used to prepare coatings (i.e. drying the peptide in the wells) did not adversely affect the activity of the peptide against the RXFP1 receptor. Notably, a 10-fold reduction in EC50 for the PLGA+B7-33 coatings was observed as compared to B7-33 alone indicating that the PLGA sequestered a portion of the peptide. This confirmed that PLGA could be used to control the release of the peptide whilst maintaining its biological activity.

### Example 4

### PLGA+B7-33 Surface Coating Preparation and Characterization

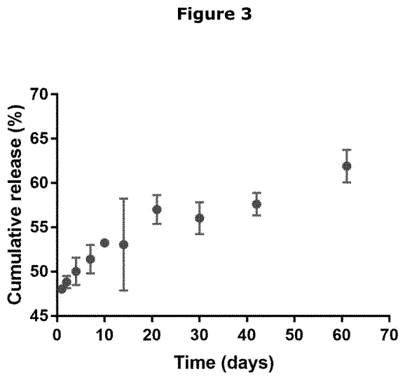

The peptide release kinetics of the PLGA+B7-33 surface coatings were assessed in the 96 well plate format using a colorimetric bicinchoninic acid (BCA) protein quantification kit (FIG. 3). During the first day of the in vitro peptide release study, an initial burst release of 48% of the peptide was observed relative to the B7-33 peptide control sample (i.e. in the absence of the polymer). Over the course of 61 days, 62% of the peptide was released from the PLGA+B7-33 surface coating corresponding to a peptide release rate of approximately 2.5 ng/day. Assuming a constant release rate of the peptide until depleted from the PLGA coating (with an initial loading of 1125 ng per well), the surface coating could potentially remain functional for approximately 240 days. The PLGA polymer control in the absence of the peptide gave a negligible signal (1%).

### Example 5

### Peptide Characterization with HPLC-MS and MALDI

In order to investigate the properties of the B7-33 peptide released from the PLGA+B7-33 surface coatings, coatings were incubated with Milli-Q water at 37° C. for 6 hours and then analysed by high performance liquid chromatography—mass spectrometry (HPLC-MS) operating in positive-ion electrospray ionisation mode. The B7-33 peptide control sample (10 μM in water) returned the molecular ion at 2985.7 Da and the associated molecular ions at 996.25, 747.44 and 598.15 m/z corresponding to the [M+3H]³⁺, [M+4H]⁴⁺, and the dominant [M+5H]⁵⁺, fragments respectively (data not shown). Interestingly, for the peptide samples released from the PLGA+B7-33 coatings and from the dried B7-33 only controls, the expected dominant [M+5H]⁵⁺ molecular ion from B7-33 at 598.15 m/z was not observed and instead the major peak was observed at 607.7 m/z. With the reasonable assumption that this peak was the [M+5H]⁵⁺ molecular ion, this corresponded to a species with molecular weight of 3033.7 Da. In order to confirm this finding, the matrix assisted laser desorption ionization (MALDI) spectra of the B7-33 control in water and B7-33 resuspended in water after drying in the well plate (i.e. dried B7-33 only control) were compared (data not shown). It was observed that the peptide prepared in water had the dominant molecular ion at 2985.7 Da as expected, however MALDI of the resuspended dried B7-33 only control exhibited four major species and the absence of the molecular ion at 2985.7 Da. The four species corresponded to oxidized species of B7-33 at [M+H]⁺+16, 32, 48, 64 which was not unexpected given the presence of methionine and tryptophan. Oxidation of the native H2 relaxin has also been documented with supporting evidence that it retains receptor recognition and importantly, as demonstrated by the earlier in vitro HEK-RXFP1 cell assay (FIG. 2), the B7-33 indeed retains activity against the RXFP1 receptor.

### Example 6

### In Vivo Subcutaneous Assessment of PLGA+B7-33 Coated Samples

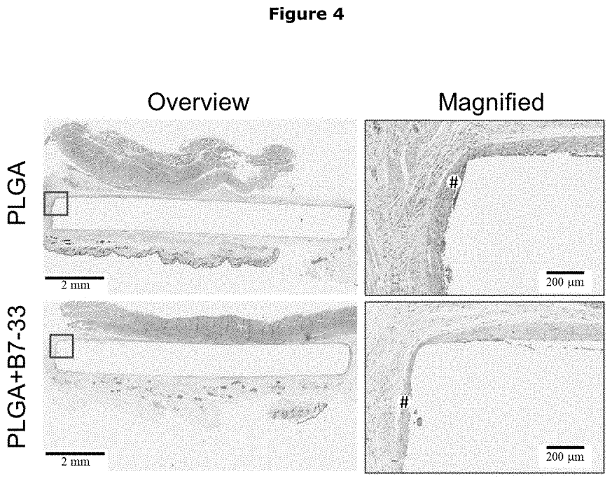

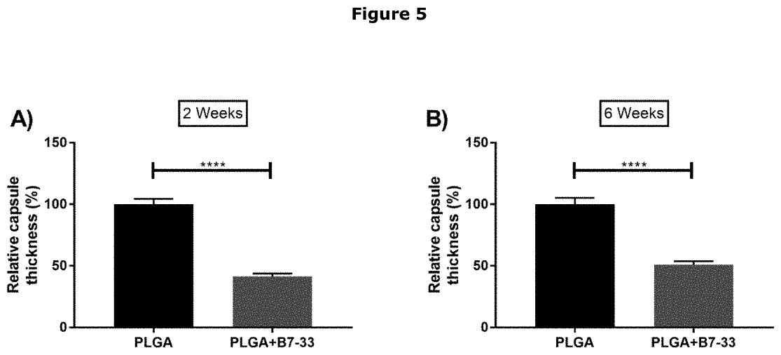

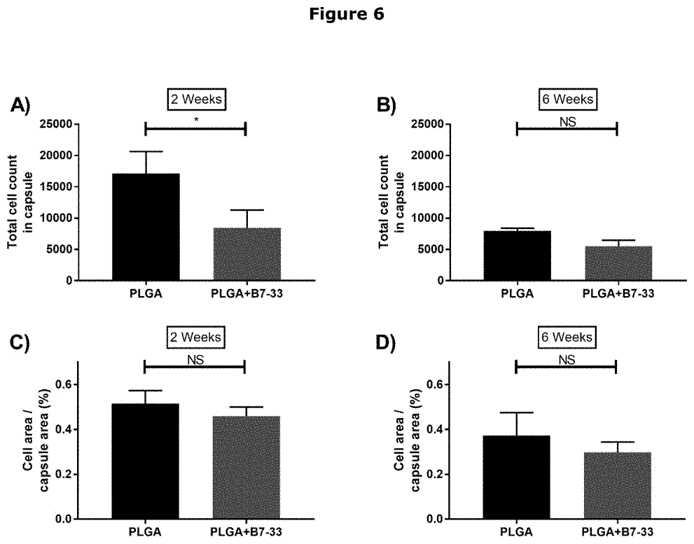

Polypropylene (PP) model implants coated with PLGA or PLGA+B7-33 were implanted subcutaneously in mice for periods of 2 and 6 weeks (within the time-frame of continuous release identified from the in vitro studies). The implants were then excised and sectioned to determine the thickness of the resulting fibrotic capsule. Microscopy images from the sectioned explants at the 6 week time point are shown in FIG. 4. Remarkably, the PLGA+B7-33 coating demonstrated a highly significant (p<0.0001) reduction in the capsule thickness relative to the PLGA coating alone (FIG. 5). At the 2 week time point, quantification of the capsule thickness revealed a decrease by 58.5% from 250.7±11.2 μm for the PLGA control, to 104.0±5.7 μm for the PLGA+B7-33 coating. For the 6 week time point, capsule thickness was reduced by 49.2% from 107.8±5.6 μm for the PLGA control, to 54.8±3.2 μm for the PLGA+B7-33 coating. Interestingly, the total cell count within the capsule also exhibited a significant reduction for the PLGA+B7-33 coatings of 50.5% at the 2 week time point, and 30.8% at the 6 week time point, as compared to the PLGA coatings alone (FIGS. 6A and 6B). The proportion of total cell area to the total fibrotic capsule area was equivalent for all samples at both time points (FIGS. 6C and 6D), though this is not unexpected as a larger cell count will produce more extracellular matrix leading to an increase in the capsule area.

### Example 7

### PLGA, PCL, or Pellethane-P80A Incorporating B7-33, Relaxin or ML290

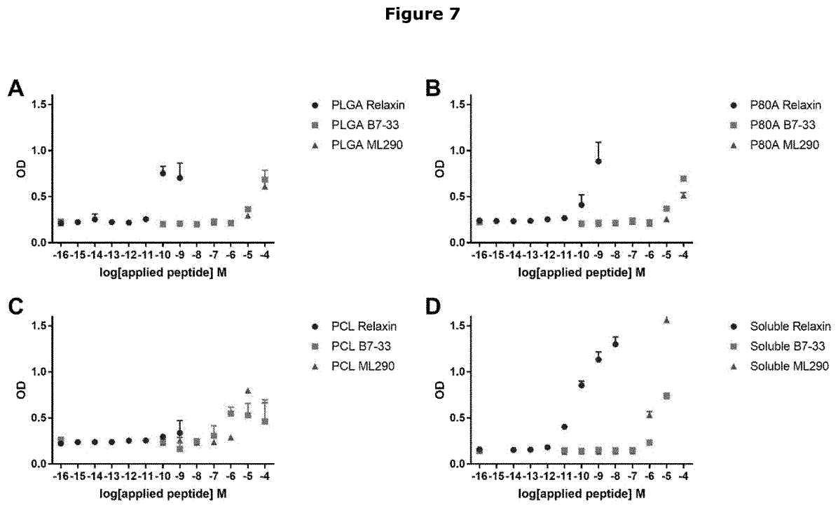

RXFP1 agonists including the whole relaxin, the relaxin peptide analogue B7-33, and the small molecule ML290 were incorporated into different polymer compositions selected from PLGA, polyurethane P80A, or PCL and prepared in 96 well plates (FIGS. 7A, B, and C, respectively). The coatings were assessed in vitro using the HEK-RXFP1 cell assay to verify biological activity at the relaxin receptor. As a control, the HEK-RXFP1 cells were seeded in tissue culture polystyrene wells and stimulated directly with each of the agonists (FIG. 7D). In each case the presence of the polymer resulted in a shift in the EC50 representing sequestering of the agonist. However, in the case of PCL containing relaxin (FIG. 7C), the signal intensity was greatly reduced, indicating that relaxin loses activity under these conditions in chloroform.

1. An implantable device, comprising a composition comprising a relaxin family peptide receptor 1 (RXFP1) agonist and a biocompatible polymer adapted to facilitate sustained release of the RXFP1 agonist from the composition in vivo, wherein:
the RXFP1 agonist is a relaxin or peptide analogue thereof comprising an amino acid sequence set forth in any one of SEQ ID NOs:2-10; and
the implantable device is configured to detect a biological parameter, provide a stimulatory signal, facilitate tissue or organ repair and/or deliver a therapeutic agent, wherein the therapeutic agent is not an RXFP1 agonist.

2. The implantable device of claim 1, wherein:
the biological parameter is selected from among an analyte, pH, temperature, light and an electrical signal; and/or the stimulatory signal is an electrical signal or an optical signal.

3. (canceled)

4. The implantable device of claim 1, wherein the implantable device comprises an electrode.

5. (canceled)

6. The implantable device of claim 1, wherein the implantable device is selected from among a biosensor, a cochlear implant, a neural implant, a peripheral nerve stimulator, a cellular implant, a nerve guide, a surgical mesh, a hernia repair implant, a retinal implant, a spinal cord stimulator, an optogenetic device, an optoelectric device, an infusion device and a drug depot.

7. (canceled)

8. The implantable device of claim 6, wherein the cellular implant comprises islet cells, mesenchymal stem cells or genetically engineered cells.

9. The implantable device of claim 1, wherein
the composition is present as a coating on at least a portion of the surface of the implantable device.

10-17. (canceled)

18. The implantable device of claim 1, wherein the biocompatible polymer comprises a biodegradable polymer.

19. The implantable device of claim 1, wherein the biocompatible polymer comprises a non-biodegradable polymer.

20. The implantable device of claim 1, wherein the biocompatible polymer comprises poly(lactic-co-glycolic acid) (PLGA), poly(ethylene glycol) (PEG), poly(ethylene oxide) (PEO), polylactide-polyglycolide homo- or co-polymers, poly(orthoester), polyglycolic acid (PGA), polylactic acid (PLA), polyurethane (PU), polyester, polycaprolactone (PCL), poly(hydroxy ethyl methacrylate), (PHEMA), polymethyl methacrylate (PMMA), polyethylene terephthalate (PET), poly(vinyl alcohol) (PVA), polysulfone, polytetrafluoroethylene (PTFE), poly(ethylene-co-vinyl acetate), polyethylene (PE), poly(propylene) (PP), poly(vinyl)chloride (PVC), polyetheretherketone (PEEK), polyvinylpyrollidone, polyacrylates, polymethacrylates, silicone, collagen, hyaluronic acid, polyanhydride, alginate, gelatin, albumin, collagen, polyamino acids, poly(amino alcohols), polyacrylamides, polymethacrylamides, zwitterionic polymers, or co-polymers or combinations of any of the foregoing.

21. (canceled)

22. The implantable device of claim 1, wherein composition comprises microspheres comprising the RXFP1 agonist and the biocompatible polymer.

23. The implantable device of claim 1, wherein the composition further comprises an additional biologically active agent.

24. (canceled)

25. The implantable device of claim 1, wherein the functionality of the implantable device in vivo is prolonged by at least about 20% compared to the functionality of a corresponding device that does not comprise the composition.

26. (canceled)

27. The implantable device of claim 1, wherein release of the RXFP1 agonist from the composition when the implantable device is implanted into a subject inhibits a foreign body response (FBR).

28. The implantable device of claim 27, wherein the FBR is inhibited by at least about 20% when compared to the FBR when a corresponding implantable device that does not comprise the composition is implanted into a subject.

29. The implantable device of claim 1, wherein release of the RXFP1 agonist from the composition when the implantable device is implanted into a subject inhibits fibrotic encapsulation of all or a portion of the implantable device, wherein the fibrotic encapsulation is inhibited by at least about 20% when compared to the fibrotic encapsulation of a corresponding implantable device that does not comprise the composition.

30. (canceled)

31. A method for preparing a device for implantation in a subject, the method comprising providing an implantable device and coating at least a portion of the surface of the device with a composition, wherein:
the composition comprises a relaxin family peptide receptor 1 (RXFP1) agonist and a biocompatible polymer adapted to facilitate sustained release of the RXFP1 agonist from the composition in vivo, wherein the RXFP1 agonist is a relaxin or peptide analogue thereof comprising an amino acid sequence set forth in any one of SEQ ID NOs:2-10; and
the implantable device is configured to detect a biological parameter, provide a stimulatory signal, facilitate tissue and/or organ repair, and/or deliver a therapeutic agent, wherein the therapeutic agent is not an RXFP1 agonist.

32. The method of claim 31, wherein:
the biological parameter is selected from among an analyte, pH, temperature, light and an electrical signal; and/or the stimulatory signal is an electrical signal or an optical signal.

33. (canceled)

34. The method of claim 31, wherein the implantable device comprises an electrode.

35. (canceled)

36. The method of claim 31, wherein the implantable device is selected from among a biosensor, a cochlear implant, a neural implant, a peripheral nerve stimulator, a cellular implant, a nerve guide, a surgical mesh, a hernia repair implant, a retinal implant, a spinal cord stimulator, an optogenetic device, an optoelectric device, an infusion device and a drug depot.

37. (canceled)

38. The method of claim 31, wherein the coating comprises spray coating, dip coating, and/or mold coating.
